(19) United States
(12) Patent Application Publication
(10) Pub. No.: US 2007/0202188 A1
(43) Pub. Date: Aug. 30, 2007

(54) Use of alkene carboxlic acid n alkylamides as flavouring agents

(76) Inventors: Jakob Ley, Holzminden (DE); Gunter Kindel, Hoxter (DE); Gerhard Krammer, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/577,947

(22) PCT Filed: Oct. 28, 2004
(86) PCT No.: PCT/EP04/52683

§ 371 Date: Mar. 26, 2007

(30) Foreign Application Priority Data
Nov. 4, 2003 (DE) 103 51 422.8

(51) Int. Cl. A61K 35/12

(52) U.S. Cl. 424/520

(57) ABSTRACT

The use of an alkenecarboxylic acid N-alkylamide of the formula 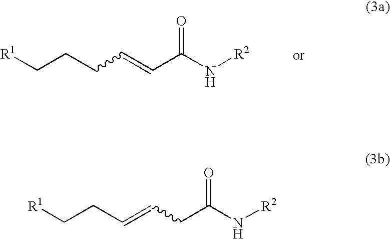 or of a mixture of two or more compounds of the formula (3a) and/or (3b), wherein, in each case

R¹ represents an alkyl radical and

R² represents a lower alkyl radical, as (i) a piquant substance and/or (ii) for generating a sensation of heat on consumption and/or (iii) for intensifying the flavour of ethanol and/or (iv) for imitating the flavour of ethanol, is described.

The invention describes the use of alkenecarboxylic acid N-alkylamides and stereoisomers thereof as intensive piquant substances and aroma substances, preferably in formulations for nutrition, oral hygiene or consumption for pleasure. The invention also relates to formulations for nutrition, oral hygiene or consumption for pleasure comprising the alkenecarboxylic acid N-alkylamides according to the invention.

Capsaicin [N-(4-hydroxy-3-methoxybenzyl)-8-methyl-(6E)-nonenoic acid amid, formula (1)] and other capsaicinoids have already been known since 1871 as piquant-tasting and heat-generating aroma substance from various capsicum species, in particular chilli. Heat-generating substances or substances having a heat-generating effect are understood as meaning those which cause a sensorial impression of heat. At an appropriately low dosage of the capsaicinoids (the threshold value lies at a dilution of approx. 1:10⁵), only a pleasant, neutral piquant taste and a sensation of heat is perceived in the mouth. A problem of capsaicin is the high acute toxicity ((LD₅₀ (mouse oral) 47 mg), which makes usability during formulation difficult, and the chronic gastritis and kidney and liver damage which occur with frequent use and overdose (Römpp Lexikon Naturstoffchemie [Römpp Dictionary of Natural Substance Chemistry], Thieme 1997, p. 109). In spite of the good sensorial properties, there is therefore a need for piquant substances which are less problematic. The piperine (1-piperoylpiperidine, formula (2)) which occurs in white pepper indeed also causes a piquant impression (Römpp Lexikon Naturstoffchemie [Römpp Dictionary of Natural Substance Chemistry], Thieme 1997, p. 500), but shows only a relative piquant taste of only approx. 1% compared with capsaicin. Piperine moreover has an intensive intrinsic taste reminiscent of pepper, so that in many formulations it can be used only with limitations.

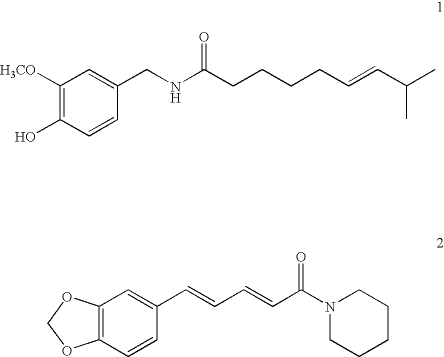

The object of the present invention was to identify substances having a piquant, tingling, mouth-watering and/or heat-generating effect and an otherwise relatively neutral aroma profile, which can be used as aroma substances in formulations for nutrition or consumption for pleasure.

The invention achieves the object described by the use of an alkenecarboxylic acid N-alkylamide of the formula

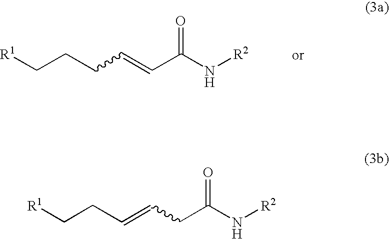

or of a mixture of two or more compounds of the formula (3a) and/or (3b)

wherein, in each case

R¹ represents an alkyl radical

and

R² represents a lower alkyl radical,

as (i) a piquant substance and/or (ii) for generating a sensation of heat on consumption, regardless of the temperature of the alkenecarboxylic acid N-alkylamide, and/or (iii) for intensifying the flavour of ethanol and/or (iv) for imitating the flavour of ethanol and/or (v) for inducing salivation, in particular for use in formulations for nutrition, oral hygiene or consumption for pleasure.

In this context, the wavy line in formulae (3a) and (3b) means that the associated double bond has the E or Z configuration. If asymmetric carbon atoms are present, the compounds of the formulae (3a) and (3b) can be in the form of pure enantiomers and/or diastereomers or mixtures thereof. Alkyl radicals in the context of the invention are linear, branched or cyclic alkyl groups having 1 to 8 carbon atoms, the following groups being preferred: ethyl, propyl, butyl, pentyl and hexyl.

Lower alkyl radicals in the context of the invention are linear, branched or cyclic alkyl groups having 1 to 5 carbon atoms, the following groups being preferred: methyl, ethyl, propyl, 2-propyl, cyclopropyl, butyl, 2-butyl, 3-methylpropyl (i.e. isobutyl), cyclobutyl, 1- or 2-methylcyclopropyl, 2-methylpropyl, pentyl, 2-pentyl, 3-pentyl, 2-methylbutyl, 3-methylbutyl, cyclopentyl and 1-, 2- or 3-methylcyclobutyl, but in particular isobutyl or 2-methylbutyl.

It is indeed already known that some fatty acid isobutylamides (“alkamides”) can cause trigeminal stimuli, such as local anaesthesia, a piquant taste or tingling in the oral cavity or on the skin and mucous membranes. Nevertheless, pellitorine (2E,4E-decadienoic acid N-isobutylamide), a substance which also quite similar structurally to the compounds of the formula (3a) and (3b), chiefly shows an anaesthetizing effect (c.f. H. C. F. Su and R. Horvat, J. Agric. Food Chem. year 1981, vol. 29, pages 115-118), which moreover starts with a delay. No sensorial data have been described hitherto for 2E,4Z-decadienoic acid N-isobutylamide, which is likewise known. In a study which is not described in more detail (B. Bryant and 1. Mezine, in ACS Symposium Series, vol. 825, Chemistry of Taste, P. Given and D. Paredes, ed., vol. 825, American Chemical Society 2002, p. 202-212), it was demonstrated that, in contrast to 2,4-alkadienoic acid amides, 2E-decenoic acid N-isobutylamide does not impart a piquant impression (no further sensorial data were published). In a study on 2E-tetradecenoic acid N-isobutylamide, likewise no piquant taste was found in the concentrations tested (H. Shibuya et al., Chem. Pharm. Bull., year 1992, volume 40, 2325-2330).

It was therefore surprising and not foreseeable for the person skilled in the art that the alkenecarboxylic acid N-alkylamides to be employed according to the invention cause a piquant, but above all hot, in some cases slightly anaesthetizing, in some cases also tingling flavour impression which starts with somewhat of a delay, and in some cases are saliva-promoting, and the sensorial impression are relatively intense at higher use concentrations and last for a relatively long time. In this context, further sensorial impressions which round off the profile are to be detected, so that the compounds of the formula (3a) and (3b) (and mixtures thereof) can be employed in an outstanding manner as aroma substances (for the purposes mentioned).

Some of the compounds of the formula 3a are known: thus, 2E-decenoic acid N-isobutylamide in Piper guineense, a plant used as a spice in Nigeria, has been found (S. K. Adesina, A. S. Adebayo, S. K. O. Adesina and R. Groening, Pharmazie 2002, 57 (9), 622-627).

3E-Decenoic acid N-isobutylamide has already been prepared and characterized earlier (c.f. Journal of the Indian Chemical Society, vol. 14, year 1937, pages 421 and 424 and Beilstein reference no. 1778318).

The following alkenecarboxylic acid N-alkylamides are particularly preferred:

2E-decenoic acid N-isobutylamide

2Z-decenoic acid N-isobutylamide

2E-decenoic acid N-(2-methylisobutyl)amide

3E-decenoic acid N-isobutylamide

3E-nonenoic acid N-isobutylamide and mixtures thereof. The present invention also provides formulations, semi-finished goods and odoriferous, aroma and flavouring substance compositions comprising the alkenecarboxylic acid N-alkylamides according to the invention. In this context, see below.

The alkenecarboxylic acid N-alkylamides (or mixture thereof) to be employed according to the invention can also be used in cosmetic or dermatological formulations for generating a sensation of heat on the skin.

In a particularly preferred embodiment of the invention, the alkenecarboxylic acid N-alkylamides (or mixtures thereof) according to the invention are used in combination with other piquant-tasting and/or heat sensation-generating and/or salivation-inducing substances or (in particular) piquant-tasting and/or salivation-inducing plant extracts. A particularly rounded-off sensorial profile can be achieved in this manner. In particular, the combination of the alkenecarboxylic acid N-alkylamides to be employed according to the invention with a piquant-tasting and/or salivation-inducing plant extract in a ratio of from 0.01:1 to 100:1, preferably 0.1:1 to 10:1, generates a pleasant sensorial profile.

Other piquant-tasting and/or heat sensation-generating substances which are suitable for combination here are e.g.: capsaicin, dihydrocapsaicin, gingerols, paradols, shogaols, piperine, carboxylic acid N-vanillylamides, in particular nonanoic acid N-vanillylamide, pellitorine or spilanthol, 2-nonenoic acid N-4-hydroxy-3-methoxyphenylamide, alkyl ethers of 4-hydroxy-3-methoxybenzyl alcohol, in particular 4-hydroxy-3-methoxybenzyl n-butyl ether, alkyl ethers of 4-acyloxy-3-methoxybenzyl alcohol, in particular 4-acetyloxy-3-methoxybenzyl n-butyl ether and 4-acetyloxy-3-methoxybenzyl n-hexyl ether, alkyl ethers of 3-hydroxy 4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylenedioxybenzyl alcohol, (4-hydroxy-3-methoxyphenyl)acetic acid amides, in particular (4-hydroxy-3-methoxyphenyl)acetic acid N-n-octylamide, vanillomandelic acid alkylamides, ferulic acid phenethylamides, nicotinaldehyde, methyl nicotinate, propyl nicotinate, 2-butoxyethyl nicotinate, benzyl nicotinate, 1-acetoxychavicol, polygodial and isodrimeninol.

Piquant-tasting plant extracts which are suitable for combination are all the plant extracts which are suitable for nutrition and cause a piquant and/or hot sensorial impression. Preferred plant extracts in this respect are, for example, pepper extract (Piper ssp., in particular Piper nigrum), water pepper extract (Polygonum ssp., in particular Polygonum hydropiper), extracts from Allium ssp. (in particular onion and garlic extracts), extracts from radish (Raphanus ssp.), horseradish extracts (Cochlearia armoracia), extracts from black (Brassica nigra), wild or yellow mustard (Sinapis ssp., in particular Sinapis arvensis and Sinapis alba), pyrethrum root extracts (Ancyclus ssp., in particular Anacylcus pyrethrum L.), cone flower extracts (Echinaceae ssp.), extracts from szechuan pepper (Zanthoxylum ssp., in particular Zanthoxylum piperitum), Spilanthes extract (Spilanthes ssp., in particular Spilanthes acmella), chilli extract (Capsicum ssp., in particular Capsicum frutescens), paradise seed extract (Aframomum ssp., in particular Aframomum melegueta [Rose] K. Schum.), ginger extract (Zingiber ssp., in particular Zingiber officinale), galanga extract (Kaempferia galanga or Alpinia galanga) and jaborandi extract (Pilocarpus species, in particular Pilocarpus jaborandi).

Salivation-inducing substances can be, for example, certain unsaturated alkamides (e.g. pellitorines, spilanthols, shogaols), alkaloids (e.g. pilocarpine), salivation-promoting peptides (e.g. substance P, tachykinins, physalaemin), and also simple fruit acids (e.g. citric acid, tartaric acid).

The salivation-inducing plant extracts can be, for example, the abovementioned plants or plant extracts containing the abovementioned salivation-inducing substances.

The piquant-tasting and/or salivation-inducing plant extracts can often be obtained from the corresponding fresh or dried plants or plant parts, but in particular from white, green or black peppercorns, water peppercorns, onions and garlic, radish root, horseradish, mustard seeds, cone flower roots, pyrethrum root, plant parts of the Zanthoxylum species, plant parts of the Spilanthes species, chilli pods, paradise seeds or ginger or galanga roots. In this context, the dried plant parts, which have preferably been comminuted beforehand, are conventionally extracted with a solvent which is suitable for foodstuffs and compositions for consumption for pleasure, at a temperature in the range of from 0° C. up to the boiling point of the particular solvent, the extract is then filtered and the filtrate is concentrated to dryness or partly concentrated, preferably by distillation or freeze or spray drying. The crude extract obtained in this way can then be worked up still further, for example treated with water vapour under pressures of from 0.01 mbar to normal pressure and/or taken up in a solvent which is suitable for foodstuffs and compositions for consumption for pleasure. Solvents which are suitable for foodstuffs and compositions for consumption for pleasure are, for example: water, ethanol, methanol, propylene glycol, glycerol, acetone, methylene chloride, diethyl ether, hexane, heptane, triacetin, a plant oil or fat, supercritical carbon dioxide or a mixture of the abovementioned solvents.

In a further particularly preferred embodiment of the invention, the alkenecarboxylic acid N-alkylamides (or mixtures thereof) to be employed according to the invention are used in combination with one or more substances which cause a physiological cooling action.

Substances which cause a physiological cooling action can be, for example, menthol and menthol derivatives (e.g. L-menthol, rac. menthol) menthyl ethers (e.g. (1-menthoxy)-1,2-propanediol, (1-menthoxy)-2-methyl-1,2-propanediol, menthyl methyl ether), menthyl esters (e.g. menthyl acetate, menthyl isobutyrate, menthyl lactate, menthyl (2-methoxy)acetate, menthyl (2-methoxyethoxy)acetate, menthyl pyroglutamate), menthyl carbonates (e.g. menthyl propylene glycol carbonate, menthyl ethylene glycol carbonate, menthyl glycerol carbonate), the half esters of menthols with dicarboxylic acid (e.g. menthyl succinate, menthyl glutarate), menthanecarboxylic acid amides (e.g. menthanecarboxylic acid N-ethylamide), menthone and menthone derivatives (e.g. menthone glycerol ketal), 2,3-dimethyl-2-(2-propyl)-butanoic acid derivative (e.g. 2,3-dimethyl-2-(2-propyl)-butanoic acid N-methylamide), isopulegol or its esters (I-(−)-isopulegol, I-(−)-isopulegol acetate), menthane derivatives (e.g. p-menthane-3,8-diol), cubebol, pyrrolidone derivatives of cycloalkyldione derivatives (e.g. 3-methyl-2(1-pyrrolidinyl)-2-cyclopenten-1-one) or icilin.

If they occur in nature, the substances which cause a physiological cooling action, also in the form of an extract, a distillate, a crystallization product or an otherwise processed and/or purified formulation, can of course originate from natural sources, preferably from naturally occurring or cultured plants or plant parts and/or plant callus or cell cultures or from fermentative processes. For example, suitable menthol-, menthone- or isopulegol-containing formulations and derivatives thereof can be obtained from a large number of Mentha ssp. by steam distillation or other distillation processes.

The present invention also provides formulations for nutrition or consumption for pleasure, comprising an active (for achieving a sensation of a piquant taste or heat, or for intensifying or imitating the flavour of ethanol, or for inducing salivation) amount of an alkenecarboxylic acid N-alkylamide of the formula (3a) or (3b) or a mixture of two or more compounds of the formula (3a) and/or (3b)

wherein, in each case

R¹

and

R² have the abovementioned meanings,

and optionally other conventional base substances, auxiliary substances and additives for foodstuffs and compositions for consumption for pleasure. These formulations as a rule comprise 0.0000001 wt. % to 10 wt. %, preferably 0.00001 to 1 wt. %, but particularly preferably 0.00001 wt. % to 0.1 wt. %, based on the total weight of the formulation, of one or more alkenecarboxylic acid N-alkylamides of the formula (3a) or (3b). Further conventional base substances, auxiliary substances and additives for foodstuffs or compositions for consumption for pleasure can be present in amounts of from 0.0000001 to 99.9999999 wt. %, preferably 10 to 80 wt. %, based on the total weight of the formulation. The formulations can furthermore comprise water in an amount of up to 99.9999999 wt. %, preferably 5 to 80 wt. %, based on the total weight of the formulation.

Preferably, the formulation according to the invention comprises at least one further piquant-tasting and/or heat sensation-generating and/or salivation-inducing substance or at least one piquant-tasting and/or salivation-inducing plant extract. In the context of the invention, the formulations for nutrition or consumption for pleasure are e.g. baked goods (e.g. bread, dry biscuits, cakes, other baked products), confectionery (e.g. chocolate, chocolate bar products, other bar products, fruit gum, hard and soft caramels, chewing gum), alcoholic or non-alcoholic drinks (e.g. coffee, tea, wine, wine-containing drinks, beer, beer-containing drinks, liqueurs, schnapps, brandies, fruit-containing carbonated drinks, isotonic drinks, refreshing drinks, nectars, fruit and vegetable juices, fruit or vegetable juice formulations), instant drinks (e.g. instant cocoa drinks, instant tea drinks, instant coffee drinks), meat products (e.g. ham, fresh sausage or uncooked sausage formulations, seasoned or marinated fresh or salted meat products), eggs or egg products (dried egg, egg white, egg yolk), cereal products (e.g. breakfast cereals, muesli bars, precooked ready-made rice products), dairy products (e.g. milk drinks, milk ice, yoghurt, kefir, fresh cheese, soft cheese, hard cheese, dried milk powder, whey, butter, buttermilk), fruit formulations (e.g. preserves, fruit-flavoured ice-cream, fruit sauces, fruit fillings), vegetable formulations (e.g. ketchup, sauces, dried vegetables, frozen vegetables, precooked vegetables, cooked vegetables), nibbles (e.g. baked or fried potato crisps or potato paste products, extrudates based on maize or peanuts), fat- and oil-based products or emulsions thereof (e.g. mayonnaise, remoulade, dressings), other ready-made dishes and soups (e.g. dried soups, instant soups, precooked soups), spices, spice mixtures and, in particular, sprinkling spices (seasonings), which are used, for example, in the snacks sector. The formulations in the context of the invention can also be used as semi-finished goods for the preparation of further formulations for nutrition or consumption for pleasure. The formulations in the context of the invention can also be in the form of capsules, tablets (non-coated and coated tablets, e.g. coatings which are resistant to gastric juice), lacquered tablets, granules, pellets, solid mixtures, dispersions in liquid phases, emulsions, powders, solutions, pastes or other formulations as food supplements which can be swallowed or chewed.

It has also proved to be particularly advantageous that the alkenecarboxylic acid N-alkylamides to be employed according to the invention, in particular in the preferred combination with piquant-tasting plant extracts, can imitate the piquant flavour of alcohol in alcoholic drinks or formulations with alcoholic drinks, and it is therefore possible for the alcohol content in alcoholic drinks or in formulations with alcoholic drinks to be adjusted to a lower level or to be replaced completely, with the same sensorial evaluation. A corresponding formulation according to the invention therefore comprises an active amount of alkenecarboxylic acid N-alkylamide of the formula

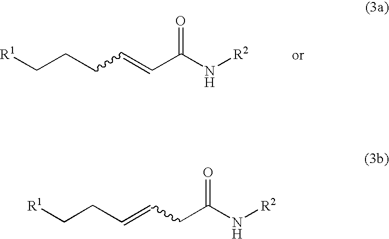

or of a mixture of two or more compounds of the formula (3a) and/or (3b),

wherein, in each case

R¹ represents an alkyl radical

and

R² represents a lower alkyl radical,

as (i) a piquant substance and/or (ii) for generating a sensation of heat on consumption, regardless of the temperature of the alkenecarboxylic acid N-alkylamide and/or (iii) for intensifying the flavour of ethanol and/or (iv) for imitating the flavour of ethanol and optionally an active amount (i) of a further piquant-tasting and/or heat-generating substance and/or (ii) of a piquant-tasting plant extract.

Preferably, in this context the flavour reminiscent of the flavour of ethanol is largely determined by the amount of alkenecarboxylic acid N-alkylamide(s). Ethanol is present in such formulations according to the invention at most in an amount of 0.5 wt. %, if an alcohol reduction compared with that in a comparison product tasting about the same is sought. Preferably, such a formulation comprises less than 0.1 wt. % ethanol.

It has proved to be particularly advantageous that the alkenecarboxylic acid N-alkylamides according to the invention can imitate the piquant taste of capsaicin, dihydrocapsaicin and nonivamide and it is therefore possible for the capsaicin content in the formulation for nutrition or consumption for pleasure to be adjusted to a considerably lower level, with the same sensorial evaluation.

The present invention also provides formulations for oral hygiene, in particular dental care compositions, such as toothpastes, tooth gels, tooth powders, mouthwashes, chewing gums and other oral care compositions, comprising an active (in this context, see above) amount of an alkenecarboxylic acid N-alkylamide of the formula (3a) or (3b)

or a mixture of two or more compounds of the formula (3a) and/or (3b),

wherein, in each case

R¹

and

R² have the abovementioned meaning

and optionally other conventional base substances, auxiliary substances and additives for such formulations. They preferably comprise 0.0000001 wt. % to 10 wt. %, preferably 0.00001 to 1 wt. %, but particularly preferably 0.00001 wt. % to 0.1 wt. %, based on the total weight of the formulation, of alkenecarboxylic acid N-alkylamides of the formula (3a) or (3b). Further conventional base substances, auxiliary substances and additives for the formulations for oral hygiene can be present in amounts of from 0.0000001 to 99.9999999 wt. %, preferably 10 to 80 wt. %, based on the total weight of the formulation. The formulations can furthermore comprise water in an amount of up to 99.9999999 wt. %, preferably 5 to 80 wt. %, based on the total weight of the formulation.

Preferably, the formulation according to the invention comprises at least one further piquant-tasting and/or heat sensation-generating and/or salivation-inducing substance or at least one piquant-tasting and/or salivation-inducing plant extract. Dental care compositions which comprise the alkenecarboxylic acid N-alkylamides according to the invention in general comprise an abrasive system (abrasive or polishing agent), such as e.g. silicas, calcium carbonates, calcium phosphates, aluminium oxides and/or hydroxylapatites, surface-active substances, such as e.g. sodium lauryl sulfate, sodium lauryl sarcosinate and/or cocamidopropylbetaine, moisture-retaining agents, such as e.g. glycerol and/or sorbitol, thickeners, such as e.g. carboxymethylcellulose, polyethylene glycols, carrageenans and/or Laponites®, sweeteners, such as e.g. saccharin, stabilizers and active compounds, such as e.g. sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetylpyridinium chloride, aluminium lactate, potassium citrate, potassium nitrate, potassium chloride, strontium chloride, hydrogen peroxide, aromas and/or sodium bicarbonate.

Chewing gums which comprise the alkenecarboxylic acid N-alkylamides to be employed according to the invention in general consist of a chewing gum base, i.e. a chewing composition which becomes plastic on chewing, of sugars of various types, sugar substitutes, sweeteners, sugar alcohols, moisture-retaining agents, thickeners, emulsifiers, aromas and stabilizers.

Preferably, the alkenecarboxylic acid N-alkylamides of the formulae (3a) and/or (3b) or formulations according to the invention can be employed in sprinkling spices, so-called seasonings, in order to avoid the dry sensation in the mouth which arises on consumption of maize, potato or rice flour crisps and snacks and to improve the sensorial flavour impression.

Preferred sprinkling spices comprise e.g. synthetic, natural or nature-identical aroma substances and carrier substances, such as e.g. maltodextrin, salts, such as e.g. sodium chloride, spices, such as e.g. paprika and pepper, sugar substances, such as e.g. saccharin, and flavour enhancers, such as e.g. monosodium glutamate and/or inosine monophosphate.

The formulations according to the invention which comprise one or more alkenecarboxylic acid N-alkylamides of the formula (3a) or (3b) can be prepared by incorporating the alkenecarboxylic acid N-alkylamide(s), as a solid, as a solution or in the form of a mixture with a solid or liquid carrier substance, into a base formulation for nutrition, oral hygiene or consumption for pleasure. Advantageously, formulations according to the invention in the form of a solution can also be converted into a solid formulation by spray drying.

According to a further preferred embodiment, for the preparation of formulations according to the invention, the alkenecarboxylic acid N-alkylamides and optionally other constituents of the formulation according to the invention can also be incorporated beforehand into emulsions, into liposomes, e.g. starting from phosphatidylcholine, into microspheres, into nanospheres or also into capsules, granules or extrudates of a matrix which is suitable for foodstuffs and compositions for consumption for pleasure, e.g. of starch, starch derivatives, cellulose or cellulose derivatives (e.g. hydroxypropylcellulose), other polysaccharides (e.g. alginate), natural fats, natural waxes (e.g. beeswax, carnauba wax) or of proteins, e.g. gelatine. In a further preferred preparation process, the alkenecarboxylic acid N-alkyl-amides are complexed beforehand with one or more suitable complexing agents, for example with cyclodextrins or cyclodextrin derivatives, preferably β-cyclodextrin, and employed in this complexed form.

A formulation according to the invention in which the matrix is chosen such that alkenecarboxylic acid N-alkylamides are released from the matrix in a delayed manner, so that a long-lasting piquant or heat-generating action is obtained, is particularly preferred.

Other constituents which can be used for the formulations according to the invention for nutrition or consumption for pleasure are further conventional base substances, auxiliary substances and additives for foodstuffs or compositions for consumption for pleasure, e.g. water, mixtures of fresh or processed, plant or animal base substances or raw materials (e.g. raw, roasted, dried, fermented, smoked and/or boiled meat, egg, bone, cartilage, fish, crustaceans and shellfish, vegetables, fruit, herbs, nuts, vegetable or fruit juices or pastes or mixtures thereof), digestible or non-digestible carbohydrates (e.g. sucrose, maltose, fructose, glucose, dextrins, amylose, amylopectin, inulin, xylans, cellulose), sugar alcohols (e.g. sorbitol, mannitol, xylitol), natural or hydrogenated fats (e.g. tallow, lard, palm fat, coconut fat, hydrogenated plant fat), fat oils (e.g. sunflower oil, groundnut oil, maize germ oil, thistle oil, olive oil, walnut oil, fish oil, soya oil, sesame oil), fatty acids or salts thereof (e.g. potassium stearate, potassium palmitate), proteinogenic or non-proteinogenic amino acids and related compounds (e.g. taurine, creatine, creatinine), peptides, native or processed proteins (e.g. gelatine), enzymes (e.g. peptidases, glucosidases, lipases), nucleic acids, nucleotides (inositol phosphate), flavour-modulating substances (e.g. sodium glutamate, 2-phenoxypropionic acid, hydroxyflavanones according to EP 1,258,200), emulsifiers (e.g. lecithins, diacylglycerols), stabilizers (e.g. carrageenan, alginate, carob bean flour, guar bean flour), preservatives (e.g. benzoic acid, sorbic acid), antioxidants (e.g. tocopherol or derivatives thereof, ascorbic acid or derivatives thereof), chelating agents (e.g. citric acid), organic or inorganic acidifying agents (e.g. malic acid, acetic acid, citric acid, tartaric acid, phosphoric acid), bitter principles (e.g. quinine, caffeine, limonin), sweeteners (e.g. saccharin, cyclamate, aspartame, neotame, neohesperidin dihydrochalcone, tagatose, sucralose), mineral salts (e.g. sodium chloride, potassium chloride, magnesium chloride, sodium phosphates), substances which prevent enzymatic browning (e.g. sulfite, ascorbic acid), essential oils, plant extracts, natural or synthetic dyestuffs or coloured pigments (e.g. carotenoids, flavonoids, anthocyans, chlorophyll and derivatives thereof), spices, as well as odoriferous substances, synthetic, natural or nature-identical aroma and flavouring substances.

Preferably, formulations according to the invention also additionally comprise an aroma composition in order to round off and refine the flavour and/or smell of the formulation. Suitable aroma compositions comprise e.g. synthetic, natural or nature-identical aroma substances as well as odoriferous substances, but in particular also other piquant-tasting and/or heat-generating substances or plant extracts.

A further aspect of the invention relates to the use of the formulations according to the invention as semi-finished goods, in particular with the aim of aromatization of finished goods produced from the semi-finished goods.

The formulations according to the invention, which are preferably semi-finished goods, as a rule comprise 0.0001 wt. % to 95 wt. %, preferably 0.001 to 80 wt. %, but in particular 0.01 wt. % to 50 wt. %, based on the total weight of the formulation, of the alkenoic acid N-alkylamides to be used according to the invention and optionally one or more other flavouring and aroma substances, and optionally also various carrier and auxiliary substances or various solvents.

Semi-finished goods which are used for aromatizing finished goods and comprise the alkenoic acid N-alkylamides (or mixtures thereof) to be used according to the invention, in combination with one or more piquant-tasting and/or heat sensation-generation substances or (in particular) piquant-tasting plant extracts comprising these substances and/or in combination with other salivation-inducing substances or plant extracts comprising these substance are particularly preferred, it being possible for the semi-finished goods also to comprise various carrier and auxiliary substances and/or various solvents.

The present invention also provides the use of 2E-decenoic acid N-isobutylamide and/or 2E-decenoic acid N-(2-methylbutyl)amide as agents for promoting saliva.

### EXAMPLES

### Preparation of the alkenoic acid N-alkylamides

### Example 1

### Preparation of 2E-decenoic acid N-isobutylamide

56 g 2E-decenoic acid were dissolved in 82 ml toluene, and 44 g thionyl chloride were added at 20-23° C. The mixture was stirred overnight at 20-23° C. and subsequently heated at 40° C. for 1 h and the toluene was then distilled off under a water-pump vacuum at 40° C. 40 g of the crude 2E-decenoic acid chloride (61 g) were dissolved in 40 ml acetone and the solution was added to a solution of 17 g isobutylamine in 40 ml acetone and 100 ml sodium hydroxide solution (9.7 g NaOH in water). The oily product was separated off and recrystallized from petroleum ether, 20 g of product being obtained in the form of slightly yellowish crystal needles (purity GC: 96.5%).

¹H-NMR (CDCl₃; 400 MHz, δ): 6.82 (1H, dt, J=15.2 Hz, J=7.0 Hz, H-3), 5.78 (1H, dt, J=15.2 Hz, J=1.5 Hz, H-2), 5.65 (1H, bs, NH), 3.14 (2H, dd, J=6.85 Hz, J=6.17 Hz, H-1′), 2.16 (2H, m, J=7 Hz, J=1.5 Hz, H-4), 1.80 (1 H, m, J=6.75 Hz, H-2′), 1.44 (2H, m, J=7.19 Hz, H-5), 1.34-1.23 (8H, m, H-6, H-7, H-8, H-9), 0.92 (6H, d, J=6.78 Hz, H-3′), 0.88 (3H, t, J=6.82 Hz, H-10) ppm.

¹³C-NMR (CDCl₃; 100 MHz, δ): 165.80 (C, C-1), 144.38 (CH, C-3), 123.32 (CH, C-2), 46.75 (CH₂, C-1′), 31.98 (CH₂, C-4), 31.70 (CH₂, C-8), 29.11 (CH₂), 29.04 (CH₂), 28.54 (CH, C-2′), 28.25 (CH₂), 22.59 (CH₂), 20.10 (2 CH₃, C-3′),14.05 (CH₃, C-10) ppm.

### Example 2

### Preparation of 2E-decenoic acid N-(2-methylisobutyl)amide

Starting from 2E-decenoic acid and 2-methylbutylamine, 2E-decenoic acid N-(2-methylbutyl)amide was prepared analogously to Example 1 (purity GC: 94,2%).

¹H-NMR (CDCl₃; 400 MHz, δ): 6.83 (1H, dt, 15.2 Hz, 6.9 Hz, H-3), 5.76 (1H, dt, 15.3 Hz, 1.5 Hz, H-2), 5.45 (1H, bs, NH), 3.27 (1H, dt, 13.4 Hz, 6.1 Hz, H-1′), 3.13 (1H, ddd, 13.4 Hz, 7.2 Hz, 6.1 Hz, H-1′), 2.17 (2H, ddd, 7.1 Hz, 7.1 Hz, 1.6 Hz, H-4), 1.58 (1H, m, 6.7 Hz, H-2′), 1.48-1.35 (2H, m), 1.35-1.23 (8H, m), 1.16 (1H, m, H-3′), 0.91 (3H, t, 7.4 Hz, H4′), 0.906 (3H, d, 6.8 Hz, H-5′), 0.88 (3H, t, 6.8 Hz, H-10) ppm.

¹³C-NMR (CDCl₃; 100 MHz, δ): 166.17 (C, C-1), 144.80 (CH, C-3), 123.57 (CH, C-2), 45.11 (CH₂, C-1′), 35.02 (CH₂, C-2′), 32.04 (CH₂, C-4), 31.77 (CH₂, C-8), 29.16 (CH₂, C-5, 6 or 7), 29.10 (CH₂, C-5, 6 or 7), 28.29 (CH₂, C-5, 6 or 7), 27.03 (CH₂, C-3′), 22.64 (CH₂, C-9), 17.19 (CH₃, C-5′), 14.08 (CH₃, C10), 11.28 (CH₃, C-4′) ppm.

### Example 3

### Preparation of 3E-nonenoic acid N-isobutylamide

2.5 g 3E-nonenoic acid and 1.84 g N-hydroxysuccinimide are dissolved in 20 ml 1,4-dioxane, and a solution of 3.30 g N,N′-dicyclohexylcarbodiimide is metered in. The mixture, which becomes cloudy, is stirred overnight at 20-23° C. and filtered. The filtrate is evaporated to dryness in vacuo (4.2 g, yellow oil). The crude product is dissolved in 50 ml chloroform, and a mixture of 1.8 ml isobutylamine and 5 ml triethylamine is added. The reaction mixture is stirred at 20-23 ° C. for a further 4 h, acidified with 25 ml 10% HCl, washed with 25 ml 10% sodium carbonate solution, dried over sodium sulfate and filtered and the filtrate is concentrated in vacuo. 3.33 g of a yellowish oil are obtained (purity GC: 96.2% main product, 2E isomer 2.5%).

¹H-NMR (CDCl₃; 400 MHz, δ): 6.02 (1H, bs), 5.63 (1H, dtt, J=15.24 Hz, J=6.5 Hz, J=1.10 Hz, H-4), 5.53 (1H, dft, J=15.27Hz, J=6.83Hz, J=1.15 Hz, H-3), 3.07 (2H, dd, J=6.82 Hz, J=5.98 Hz, H-1′), 2.94 (2H, dd, J=6.8 Hz, J=1.10 Hz, H-2), 2.05 (2H, td, J=7.05 Hz, J=0.97 Hz, H-5), 1.77 (1 H, m, J=6.73 Hz, H-2′), 1.43-1.23 (6H, m, H-6, H-7, H-8), 0.90 (6H, d, J=6.7 Hz, H-3′), 0.88 (3H, t, J=6.95 Hz, H-9) ppm.

### Example 4

### Preparation of 3E-decenoic acid N-isobutylamide

2.72 g 3E-decenoic acid and 1.84 g N-hydroxysuccinimide are dissolved in 20 ml 1,4-dioxane, and a solution of 3.30 g N,N′-dicyclohexylcarbodiimide is metered in. The mixture, which becomes cloudy, is stirred overnight at 20-23° C. and filtered. The filtrate is evaporated to dryness in vacuo (yellow oil). The crude product is dissolved in 50 ml chloroform, and a mixture of 1.8 ml isobutylamine and 5 ml triethylamine is added. The reaction mixture is stirred at 20-23° C. for a further 4 h, acidified with 25 ml 10% HCl, washed with 25 ml 10% sodium carbonate solution, dried over sodium sulfate and filtered and the filtrate is concentrated in vacuo. Approx. 3 g of a yellowish oil are obtained, and are purified by chromatography on silica gel 60 with the eluents n-hexane/ethyl acetate.

¹H-NMR (CDCl₃; 400 MHz, δ): 5.70 (1H, bs), 5.64 (1H, dtt, J=15.2 Hz, J=6.6 Hz, J=1 Hz, H-4), 5.52 (1 H, dtt, J=15.2 Hz, J=7 Hz, J=1 Hz, H-3), 3.07 (2H, dd, J=6.9 Hz, J=6.1 Hz, H-1′), 2.95 (2H, dd, J=7 Hz, J=1 Hz, H-2), 2.02 (2H, td, J=7 Hz, J=1 Hz, H-5), 1.72 (1H, m, J=6.7 Hz, H-2′), 1.43-1.23 (8H, m, H-6, H-7, H-8, H-9), 0.86 (6H, d, J=6,7 Hz, H-3′), 0.85 (3H, t, J=7 Hz, H-10) ppm.

¹³C-NMR (CDCl₃; 100 MHz, δ): 171.32 (C, C-1), 136.85 (CH, C4), 122.70 (CH, C-3), 46.83 (CH₂, C-1′), 40.65 (CH₂, C-2), 32.57 (CH₂, C-5), 31.70 (CH₂, C-8), 29.20 (CH₂, C-6), 28.87 (CH₂, C-7), 28.46 (CH, C-2′), 20.04 (2□CH₃, C-3′), 14.09 (CH₃, C-10) ppm.

### Example 5

### Sensorial Evaluation

The substance to be tasted (see below) was dissolved in ethanol and the ethanolic solution was then diluted with 11% strength sugar solution (final concentration: c). For tasting, in each case approx. 5 ml of the sugar solution was swallowed. If the threshold value of the substance was known, a value just above the threshold value was chosen for the tasting. A group of 6-8 testers tasted the solutions.

a) Profile of 2E-decenoic acid N-isobutylamide (Example 1):

c=10 ppm: slightly piquant, anaesthetizing, heat sensation, saliva-promoting; slightly hereby, citrus-like, tingling, ginger-like

b) Profile of 2E-decenoic acid N-(2-methylbutyl)amide (Example 2):

c=10 ppm: slightly piquant, saliva-promoting, tingling, mild heat sensation

c) Profile of 3E-nonenoic acid N-isobutylamide (Example 3):

c=10 ppm: anaesthetizing, piquant taste builds up, impression of sweet palmond, slightly bitter

d) Profile of 3E-decenoic acid N-isobutylamide (Example 4):

c=10 ppm: tickling piquant taste, builds up slowly in the pharynx, slightly bitter

### Comparison Examples

e) Profile of dihydrocapsaicin:

c=100 ppb: action starts with a slight delay in the pharyngeal cavity, aggressive, burning piquant taste (chilli, slight evolution of heat)

f) Profile of 2E,4E-decadienoic acid N-isobutylamide (trans-pellitorine)

c=10 ppm: saliva-promoting, fatty, fruity, slightly tingling, slightly piquant

### Example 6

### Use in an Apple Schnapps as an Alcohol Flavour Enhancer

Standard formulation with 20 vol. % ethanol:

| | |
|---|---|
| 20 l | ethanol 96 vol. % |
| 5.2 l | aroma (natural apple fruit juice liqueur aroma, 15 vol. %) |
| 27 kg | sugar syrup |
| 1 kg | citric acid monohydrate |

Top up with demineralized water to 100 l; total amount 100 l

Reduced formulation with alcohol flavour enhancer

| | |
|---|---|
| 14.90 l | ethanol 96 vol. % |
| 5.2 l | aroma (natural apple fruit juice liqueur aroma, 15 vol. %, |
| | contains 0.01 wt. % 2E-decenoic acid N-isobutylamide) |
| 27 kg | sugarsyrup |
| 1 kg | citric acid monohydrate |

Top up with demineralized water to 100 l; total amount 100 l

The two formulations are virtually identical sensorially.

### Example 7

### Use in Combination with a Piquant Plant Extract as an Alcohol Flavour Enhancer

Standard formulation with 20 vol. % ethanol:

| | |
|---|---|
| 20 l | ethanol 96 vol. % |
| 5.2 l | aroma (natural apple fruit juice liqueur aroma, 15 vol. %) |
| 27 kg | sugar syrup |
| 1 kg | citric acid monohydrate |

Top up with demineralized water to 100 l; total amount 100 l

Reduced formulation with alcohol flavour enhancer

| | |
|---|---|
| 14.90 l | ethanol 96 vol. % |
| 5.2 l | aroma (natural apple fruit juice liqueur aroma, 15 vol. %, |
| | contains 0.0025 wt. % 2E-decenoic acid N-isobutylamide and |
| | 0.0075 wt. % paradise seed extract) |
| 27 kg | sugar syrup |
| 1 kg | citric acid monohydrate |

Top up with demineralized water to 100 ; l total amount 100 l

The two formulations are virtually identical sensorially.

### Example 8

### Use in an Alcohol-Free Formulation as an Alcohol Imitation

Standard formulation with alcohol (5.5 vol. % alcohol):

| | |
|---|---|
| 4.06 g | absolute ethanol, analytical grade |
| 20 g | invert sugar syrup 66.5% dry matter |
| 75.94 g | tap water |

Formulation without alcohol:

| | |
|---|---|
| 20 g | invert sugar syrup 66.5% dry matter |
| 80 g | tap water |

20 ppm, based on the total formulation, of 2E-decenoic acid N-isobutylamide from Example 1

For the individual formulations, all the ingredients are mixed and the aroma is metered in last.

Tasting: the formulation without alcohol has a piquant character of alcohol, which is in good agreement with the standard formulation (5.5 vol. %).

### Example 9

### Use in an Alcohol-Free Formulation as an Alcohol Imitation Together with a Piquant Plant Extract

Standard formulation with alcohol (5.5 vol. % alcohol):

| | |
|---|---|
| 4.06 g | absolute ethanol, analytical grade |
| 20 g | invert sugar syrup 66.5% dry matter |
| 75.94 g | tap water |

Formulation without alcohol:

| | |
|---|---|
| 20 g | invert sugar syrup 66.5% dry matter |
| 80 g | tap water |

20 ppm, based on the total formulation, of 2E-decenoic acid N-isobutylamide from Example 1

| | |
|---|---|
| 0.3 g | paradise seed extract |

For the individual formulations, all the ingredients are mixed and the aroma is metered in last.

Tasting: here also, the formulation without ethanol has a piquant character of alcohol, which is in very good agreement with the standard formulation (5.5 vol. %).

### Example 10

### Use in a Toothpaste as an Aroma Substance

| | | Amount employed |
|---|---|---|
| Part | Constituent | in wt. % |
| A | Demineralized water | 22.00 |
| | Sorbitol (70%) | 45.00 |
| | Solbrol ® M, sodium salt (Bayer AG, | 0.15 |
| | p-hydroxybenzoic acid alkyl ester) | |
| | Trisodium phosphate | 0.10 |
| | Saccharin 450 | 0.20 |
| | Sodium monofluorophosphate | 1.12 |
| | Polyethylene glycol 1500 | 5.00 |
| B | Sident 9 (abrasive silicon dioxide) | 10.00 |
| | Sident 22 S (thickening silicon dioxide) | 8.00 |
| | Sodium carboxymethylcellulose | 0.90 |
| | Titanium dioxide | 0.50 |
| C | Demineralized water | 4.53 |
| | Sodium lauryl sulfate | 1.50 |
| D | Aroma comprising 0.1% 2E-decenoic acid | 1 |
| | N-isobutylamide | |

The constituents of parts A and B are each premixed in themselves and stirred together thoroughly in vacuo at 25-30° C. for 30 min. Part C is premixed and added to A and B; D is added and the mixture is stirred thoroughly in vacuo at 25-30° C. for 30 min. After release, the toothpaste is finished and can be transferred to containers.

### Example 11

### Use in a Sugar-Free Chewing Gum as an Aroma Substance

| | | Amount employed |
|---|---|---|
| Part | Constituent | in wt. % |
| A | Chewing gum base, Company “Jagum T” | 30.00 |
| B | Sorbitol, powdered | 39.00 |
| | Isomalt ® (Palatinit GmbH) | 9.50 |
| | Xylitol | 2.00 |
| | Mannitol | 3.00 |
| | Aspartame ® | 0.10 |
| | Acesulfame ® K | 0.10 |
| | Emulgum ® (Colloides Naturels, Inc.) | 0.30 |
| C | Sorbitol, 70% | 14.00 |
| | Glycerol | 1.00 |
| D | Aroma comprising 0.1% 2E-decenoic acid | 1 |
| | N-isobutylamide | |

Parts A to D are mixed, and kneaded intensively. The crude mass can be processed e.g. in the form of thin strips to give ready-for-consumption chewing gums.

### Example 12

### Use in a Mouthwash as an Aroma Substance

| | | Content |
|---|---|---|
| Part | Constituent | (%) |
| A | Ethanol | 10.00 |
| | Cremophor ® CO 40 (BASF, detergent) | 1.00 |
| | Benzoic acid | 0.12 |
| | Aroma comprising 0.4% 2E-decenoic acid | 0.25 |
| | N-isobutylamide | |
| B | Demineralized water | 83.46 |
| | Sorbitol, 70% | 5.00 |
| | Sodium saccharin 450 | 0.07 |
| | L-Blue 5000 e.c., 1% in water (dyestuff) | 0.10 |

The constituents of parts A and B are each mixed in themselves. Part B is stirred slowly into Part A, until the mixture is homogeneous.

### Example 13

### Use in a Sprinkling Spice for Fried Baked Nibbles

A mixture of 7 g cheese dry aroma for snacks and 0.07 g 2E-decenoic acid N-isobutylamide is sprinkled over 100 g non-spiced tortilla chips.

### Example 14

### Use in a Biscuit Cream Filling

100 g standard cream filling are mixed intensively with 0.4 g strawberry aroma and 0.1 g 2E-decenoic acid N-isobutylamide.

### Example 15

### Use in a Hard Caramel as an Aroma Substance

| | Content |
|---|---|
| Constituent | (%) |
| Sucrose | 57.00% |
| Glucose syrup | 29.00% |
| Water | 13.82% |
| Aroma (comprising 50% menthol and 50% peppermint oil) | 0.1% |
| Ethanolic solution, comprising 1% 2E-decenoic acid N- | 0.08% |
| isobutylamide | |

Sucrose is dissolved in water at 115° C. The glucose syrup is added and the mixture is brought to 140° C. The aroma and the ethanolic solution are added and, after thorough mixing, the mixture poured with a temperature of 130-135° C. into moulds and left to solidify therein.

1. A method of providing or enhancing a sensorial mouth perception impression of heat by adding to a composition of an alkenecarboxylic acid N-alkylamide of the formula 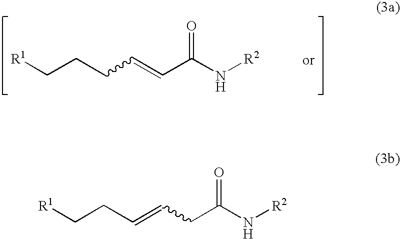
or of a mixture of two or more compounds of the formula (3b), wherein, in each case
R¹ represents an alkyl radical, and
R² represents a lower alkyl radical,
as (i) a piquant substance and/or (ii) for generating a sensation of heat on consumption, regardless of the temperature of the alkenecarboxylic acid N-alkylamide, and/or (iii) for intensifying the flavour of ethanol and/or (iv) for imitating the flavour ofethanol and/or (v) for inducing salivation.

2. A method according to claim 1 in a formulation for nutrition or consumption for pleasure.

3. A method according to claim 1 in a formulation for oral hygiene.

4. Formulation for nutrition, oral hygiene or consumption for pleasure or cosmetic or dermatological formulation, comprising an active amount of an alkenecarboxylic acid N-alkylamide of the formula 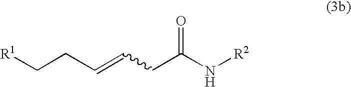
or of a mixture of two or more compounds of the formula (3a), wherein, in each case
R¹ represents an alkyl radical
and
R² represents a lower alkyl radical.

5. Formulation according to claim 4, comprising at least one further piquant-tasting and/or heat sensation-generating and/or salivation-inducing substance.

6. Formulation according to claim 4, comprising at least one piquant-tasting and/or salivation-inducing plant extract.

7. Formulation according to claim 4, comprising at least one substance which causes a physiological cooling action.

8. Formulation according to claim 4 in the form of semi-finished goods.

9. Formulation according to claim 4 in the form of odoriferous, aroma or flavouring substance compositions or a seasoning mixture.

10. Formulation having a flavour reminiscent of the flavour of ethanol, comprising an active amount of an alkenecarboxylic acid N-alkylamide of the formula 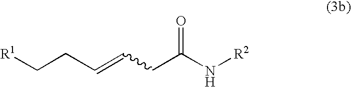
or of a mixture of two or more compounds of the formula (3a), wherein, in each case
R¹ represents an alkyl radical
and
R² represents a lower alkyl radical,
as (i) a piquant substance and/or (ii) for generating a sensation of heat on consumption, regardless of the temperature of the alkenecarboxylic acid N-alkylamide, and/or (iii) for intensifying the flavour of ethanol and/or (iv) for imitating the flavour of ethanol and/or (v) for inducing salivation, and optionally an active amount (i) of a further piquant-tasting and/or heat-generating and/or salivation-inducing substance and/or (ii) of a piquant-tasting and/or salivation-inducing plant extract.

11. Formulation according to claim 10, wherein the flavour reminiscent of the flavour of ethanol is largely determined by the amount of alkenecarboxylic acid N-alkylamide(s).

12. Formulation according to claim 10, wherein ethanol is present at most in an amount of 0.5 wt. %, based on the total weight of the formulation.

13. (canceled)

14. A method according to one of claim 1 wherein:
R¹ denotes ethyl, propyl, butyl, pentyl or hexyl, and
R² denotes methyl, ethyl, propyl, 2-propyl, cyclopropyl, butyl, 2-butyl, 3-methylpropyl, cyclobutyl, 1- or 2-methylcyclopropyl, 2-methylpropyl, pentyl, 2-pentyl, 3-pentyl, 2-methylbutyl, 3-methylbutyl, cyclopentyl or 1-, 2- or 3-methylcyclobutyl.

15. A formulation according to claim 10, wherein:
R¹ denotes propyl, and
R² denotes methyl, ethyl, propyl, 2-propyl, cyclopropyl, butyl, 2-butyl, 3-methylpropyl, cyclobutyl, 1- or 2-methylcyclopropyl, 2-methylpropyl, pentyl, 2-pentyl, 3-pentyl, 2-methylbutyl, 3-methylbutyl, cyclopentyl or 1-, 2- or 3-methylcyclobutyl, or
R¹ denotes ethyl, propyl, butyl, pentyl or hexyl, and
R² denotes ethyl or cyclopropyl.

16. A method according to claim 14, wherein:
R¹ denotes propyl and R² denotes ethyl, or
R¹ denotes propyl and R² denotes cyclopropyl.

17. Compound of the formula (3b) 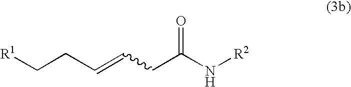
wherein R¹ represents an alkyl radical, and R² represents a lower alkyl radical, with the proviso that said compound is not 3E-decenoic acid N-isobutylamide.

18. Compound according to claim 16, wherein
R¹ denotes ethyl, propyl, butyl, pentyl or hexyl, and
R² denotes methyl, ethyl, propyl, 2-propyl, cyclopropyl, butyl, 2-butyl, 3-methylpropyl, cyclobutyl, 1- or 2-methylcyclopropyl, 2-methylpropyl, pentyl, 2-pentyl, 3-pentyl, 2-methylbutyl, 3-methylbutyl, cyclopentyl or 1-, 2- or 3-methylcyclobutyl.

19. Compound according to claim 17, wherein
R¹ denotes propyl, and
R² denotes methyl, ethyl, propyl, 2-propyl, cyclopropyl, butyl, 2-butyl, 3-methylpropyl, cyclobutyl, 1- or 2-methylcyclopropyl, 2-methylpropyl, pentyl, 2-pentyl, 3-pentyl, 2-methylbutyl, 3-methylbutyl, cyclopentyl or 1-, 2- or 3-methylcyclobutyl, or
R¹ denotes ethyl, propyl, butyl, pentyl or hexyl,
R² denotes ethyl or cyclopropyl.

20. Compound according to one of claim 17, wherein
R¹ denotes propyl and R² denotes ethyl, or
R¹ denotes propyl and R² denotes cyclopropyl.
